(19) United States
(12) Patent Application Publication
(10) Pub. No.: US 2007/0136897 A1
(43) Pub. Date: Jun. 14, 2007

(54) Nucleic acid molecules encoding hyperactive nucleoside di-phosphate kinase 2 and uses thereof

(76) Inventors: Jeong-II Kim, Gwangsan-gu (KR); Yun-Jeong Han, Gwangsan-gu (KR); Seong-Hee Kim, Jinju-shi (KR); Pill-Soon Song, Gwangsan-gu (KR)

Correspondence Address: KENYON & KENYON LLP, 1500 K STREET N.W., SUITE 700, WASHINGTON, DC, 20005, US

(21) Appl. No.: 11/247,236

(22) Filed: Oct. 12, 2005

(51) Int. Cl. A01H 1/00; C07H 21/04; C12N 5/04; C12N 15/82

(52) U.S. Cl. 800/289; 435/468; 435/419; 536/23.2

(57) ABSTRACT

The present invention includes modified Arabidopsis Nucleoside Di-Phosphate Kinase 2 (NDPK2) nucleic acid molecules whose enzymatic activity have been increased (i.e. hyperactive). NDPKs are ubiquitous housekeeping enzymes that catalyze the transfer of γ-phosphoryl group from a nucleoside triphosphate (NTP) to a nucleoside diphosphate (NDP), and also multifunctional proteins that regulate a variety of eukaryotic cellular activities, including cell proliferation, development, and differentiation. In plants, NDPKs are reported to play a key role in the signaling of both stress and light. Among three NDPKs (NDPK1, NDPK2, NDPK3) in a model plant, Arabidopsis thaliana, NDPK2 was reported as a positive signal transducer of phytochrome-mediated plant light signaling and to regulate cellular redox state, which enhances multiple stress tolerance in transgenic plants. Thus, the plants with the hyperactive NDPK2 are expected to possess higher efficiency of light utilization and enhanced tolerance to various environmental stresses such as cold, salt, and oxidative stresses. Since abiotic stress is one of the most important factors to limit the productivity of many crops, the hyperactive NDPK2 can be used for the development of high-yielding multiple stress tolerant plants with higher efficiency of light utilization. In this invention, several hyperactive NDPK2 were generated by C-terminal deletion and site-directed mutagenesis. Therefore, the present invention can be utilized to develop multiple stress tolerant and efficiently light-utilizing plants, which can eventually increase crop yields. The invention also includes plants having at least one cell expressing the modified NDPK2, vectors comprising at least one portion of the modified NDPK2 nucleic acids, and methods using such vectors for producing plants with enhanced light sensitivity and stress tolerance.

### BACKGROUND OF THE INVENTION

1. Field of the invention

This invention relates to a nucleoside di-phosphate kinase 2 (NDPK2) whose enzymatic activity has been increased (i.e. hyperactive), giving plants higher efficiency of light utilization and enhanced stress tolerance that can increase crop yields. The NDPK2 functions in catalyzing the transfer of a γ-phosphoryl group from a nucleoside triphosphate (NTP) to a nucleoside diphosphate (NDP), and also in the signaling of both stress and light in plants. The hyperactive NDPK2, as a positive signal transducer of phytochromes, can positively mediate plant light signaling, which make plants to use light more efficiently for their productivity. The hyperactive NDPK2 can also enhance multiple stress tolerance in plants, which increases crop yields. Thus, the developed hyperactive NDPK2 enable us to develop stress tolerant plants with higher efficiency of light utilization, resulting in high yields.

2. Description of Prior Art

Nucleoside diphosphate kinase (EC 2.7.4.6; NDPK) is a ubiquitous enzyme that catalyzes the transfer of the γ-phosphate from nucleoside triphosphate (NTP) to nucleoside diphosphate (NDP). Although NDPK has been considered for decades as a housekeeping enzyme to maintain nucleoside triphosphate levels in organisms, growing evidence has indicated that NDPK also participates in the regulation of growth, development and signal transduction processes. In Drosophila, mutation of Awd (a NDPK homologue) results in abnormal cell morphology. In human, Nm23-H1, a human NDPK, functions as a tumor metastasis suppressor. Additionally, Nm23-H2, an isoform of Nm23-H1, acts as a transcription factor that binds to the c-myc oncogene promoter and stimulates transcription. The ability of NDPK to supply GTP also implies a role in G-protein-mediated signaling. Recent reports suggested that NDPK could serve as a guanine nucleotide exchange factor (GEF) as well as a GTPase activating protein (GAP). Therefore, NDPK is a multifunctional enzyme.

In plants, NDPKs have been characterized in Arabidopsis, rice, oat, and pea, and NDPKs are reported to be involved in responses to heat stress, UV-B light signaling, growth, reactive oxygen species signaling, and phytochrome-mediated light signaling. Arabidopsis thaliana expresses three NDPKs, NDPK1, 2 and 3 (GenBank accession Nos. AF017641, AF017640, and AF044265, respectively), among which NDPK2 has been studied the most. NDPK2 whose amino acid sequence was known as SEQ ID NO: 1 was reported as the only NDPK among the three isoforms to interact with phytochromes, the molecular light switches that mediate the regulation of the plant's growth and development. NDPK2 is catalytically activated in the presence of biologically active Pfr phytochromes and appears to exert a positive effect on cotyledon unfolding and greening responses elicited by light and phytochromes (Choi et al., 1999). In addition, our recent results revealed that phytochrome stimulates the enzymatic activity of NDPK2 by lowering the pKₐ value of His 197 (Shen et al., 2005). Thus, Arabidopsis NDPK2 is a positive signaling component of phytochrome-mediated signal transduction pathways. Furthermore, NDPK2 has also been reported to be involved in protection against ROS (Reactive Oxygen Species) stress (Moon et al., 2003). NDPK2 interacts with two oxidative stress-activated mitogen-activated protein kinases to regulate positively in the down-regulation of the cellular redox state. Thus, overexpression of NDPK2 in plants resulted in enhanced tolerance against several environmental stresses such as cold, salt, and oxidative conditions (Moon et al., 2003).

The use of NDPK2 to increase light utilization efficiency and stress tolerance in plants is limited because of the limitation of expression levels. Moreover, both light utilization efficiency and stress tolerance are believed to relate positively with NDPK2 enzymatic activity. Thus, hyperactive NDPK2 would be ideal to improve plants' light utilization efficiency and stress tolerance for the increase of productivity. During the study of NDPK2 protein structure and enzymatic mechanisms, we obtained several hyperactive NDPK2 mutants by C-terminal deletion and site-specific mutations. Therefore, hyperactive NDPK2 in this invention can be practically applied to enhance light utilization efficiency and stress tolerance of economically important higher plants, which can increase their productivities. The plants referred to here are those economically important in agriculture and horticulture. As used herein, the term “economically important higher plants” refers to higher plants that are capable of photosynthesis and widely cultivated for commercial purpose. The term “plant cell” includes any cells derived from a higher plant, including differentiated as well as undifferentiated tissues, such as callus and plant seeds.

### SUMMARY OF THE INVENTION

The present invention relates to nucleic acid molecules encoding modified nucleoside di-phosphate kinase 2 (NDPK2) proteins whose enzymatic activity has been increased (i.e. hyperactive). Such nucleic acid molecules confer increased efficiency of light utilization and multiple stress tolerance to plants. Since efficiency of light utilization and environmental stress are each one of the most important factors to limit the productivity of many crops, the hyperactive NDPK2 can be used for the development of higher light utilizing and multiple stress tolerant plants with higher yields. In this invention, several hyperactive NDPK2 were generated by C-terminal deletion and site-directed mutagenesis, and their enzymatic activities were characterized. Therefore, this invention relates to the development of hyperactive NDPK2 and their application to develop efficiently light-utilizing and stress-tolerant plants with high-yields.

### BRIEF DESCRIPTION OF THE DRAWINGS

FIG. 1 shows in vitro binding between phytochrome and NDPK2. Immunoprecipitation of purified native oat phytochrome A (phyA) and NDPK2 incorporated with ¹⁴C-labeled methionine were performed. 105 nM phytochrome was used in each reaction. The Pfr form of phytochrome (♦) binds NDPK2 preferentially. NDPK2 saturation binding was reached at ˜330 nM, and 100% interaction was assumed. Pr+NK2+dCDP (▪); Pfr+NK2+dCDP (♦); Pr+NK2 (▴); Pfr+NK2 (●).

FIG. 2 shows the phytochrome interaction and enzymatic activities of C-terminal deletion NDPK2 mutants. (A) Immunoprecipitation between native oat phyA and NDPK2 C-terminal deletion mutants. L225Stop binds Pfr phytochrome well. K214Stop almost has no binding with Pfr phytochrome. NDPK2 kinase site mutant H197C and Kpn loop mutant P175S bind Pfr phytochrome equal to the wild type. (B) The stimulation of Pfr phytochrome on NDPK2 C-terminal deletion mutants. 145 nM and 290 nM Pfr phytochrome were tested in this assay, respectively. Pfr phytochrome is not able to stimulate K214Stop in the γ-phosphate exchange activity assay. These results indicate that the extreme C-terminal fragment of NDPK2 is the phytochrome binding site.

FIG. 3 shows the γ-phosphate exchange activities of C-terminal deletion mutants. R230Stop, L225Stop and K214Stop show higher activity than the wild type, while N204Stop and S199Stop show lower activity than the wild type. * represents hyperactive NDPK2.

FIG. 4 shows the γ-phosphate exchange activities of H197-surrounding residue mutants. Y87D shows a higher activity than the wild type. Other mutants have a lower activity than the wild type. * represents hyperactive NDPK2.

### DETAILED DESCRIPTION OF THE INVENTION

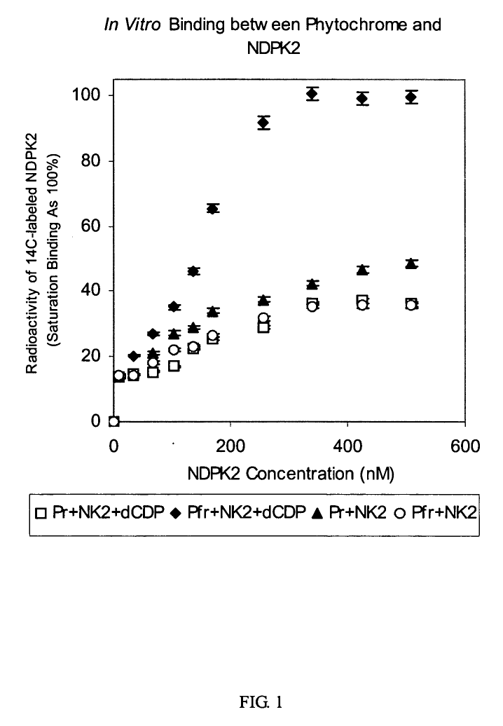

It is previously reported that NDPK2 interacts with the C-terminal domain of Arabidopsis phyA in yeast two-hybrid screening, and can be catalytically stimulated by the Pfr form of native oat phyA (Choi et al., 1999). To better understand the interaction between NDPK2 and phytochrome, an in vitro binding experiment was conducted. Purified native oat phyA was tested in the immunoprecipitation reaction with NDPK2. NDPK2 protein incorporated with ¹⁴C-labeled methionine was expressed and tested in a binding assay with native oat phyA. Results confirmed the Pfr-preferred interaction between phytochrome and NDPK2 in the presence of dCDP (FIG. 1). In addition, results also indicated that the saturation binding of NDPK2 occurred at approximately 330 nM when 105 nM phytochrome was tested. Thus, the binding ratio between phytochrome and NDPK2 is close to the ratio of phytochrome dimer to NDPK2 hexamer.

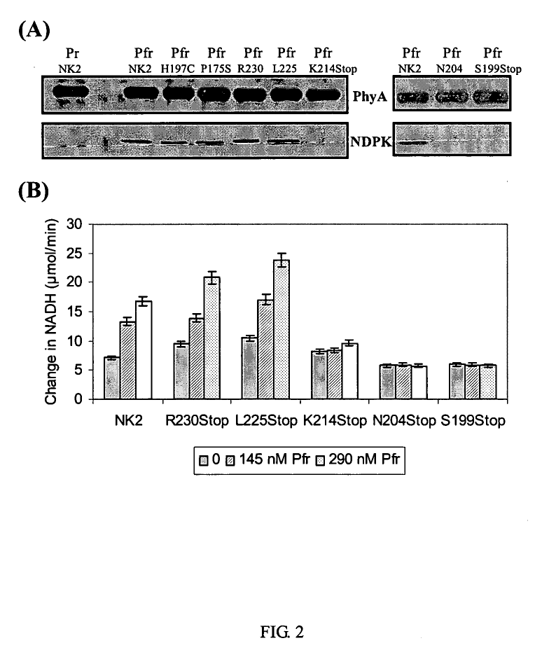

In studying the phytochrome binding site in NDPK2, NDPK2 mutants, including the C-terminal deletion mutants, kinase site mutant H197C, and Kpn loop mutant P175S, were made and tested in the binding assays with the Pfr form of native oat phyA (FIG. 2A). Results indicated that mutants H197C and P175S interacted with Pfr phytochrome as well as the wild type, suggesting that the kinase activity and Kpn loop of NDPK2 are not involved in the binding with phytochrome directly. Furthermore, the C-terminal deletion mutants R230Stop (SEQ ID NO: 2) and L225Stop (SEQ ID NO: 3) interacted with Pfr phytochrome well, whereas mutant K214Stop (SEQ ID NO: 4) showed almost no interaction with Pfr phytochrome. Moreover, no interactions between Pfr phytochrome and mutants N204Stop and S199Stop were observed. These results indicated that the NDPK2 C-terminal fragment, especially residues 214-224, is critical in the interaction with phytochrome. Results from the γ-phosphate exchange activity assay were consistent with the in vitro binding assay. Mutants R230Stop and L225Stop were stimulated by Pfr phytochrome significantly, whereas mutants K214Stop, N204Stop, and S199Stop were not stimulated under the identical conditions (FIG. 2B), suggesting the C-terminal fragment of NDPK2 as the phytochrome-binding site. Thus, R230Stop and L225Stop were hyperactive NDPK2 that binds normally to its upstream signaling molecule, phytochrome. To investigate the mechanism of hyperactivity of these NDPK2 mutants, the nucleotide affinity was investigated. By using two natural nucleotide substrates of NDPK2, ATP and GDP, the Kₘ values were measured to examine the relationship between the nucleotide affinity and NDPK activity in NDPK2 hyperactive mutants (Table 1). Both mutants showed decreased Kₘ values: 0.211 mM and 0.213 mM for ATP, and 0.203 mM and 0.191 mM for GDP. Therefore, it appears that a higher nucleotide affinity corresponds to a higher NDPK activity.

TABLE 1
The Kₘ Values of NDPK2, Pfr Phytochrome-Stimulated
NDPK2, and NDPK2 C-terminal Deletion Mutants
Determined by Using ATP and GDP.

| | ATP | GDP |
|---|---|---|
| Kₘ | (dCDP as acceptor) | (ATP as donor) |
| NDPK2 (SEQ ID NO: 1) | 0.286 mM | 0.231 mM |
| R230Stop (SEQ ID NO: 2) | 0.211 mM | 0.203 mM |
| L225Stop (SEQ ID NO: 3) | 0.213 mM | 0.191 mM |

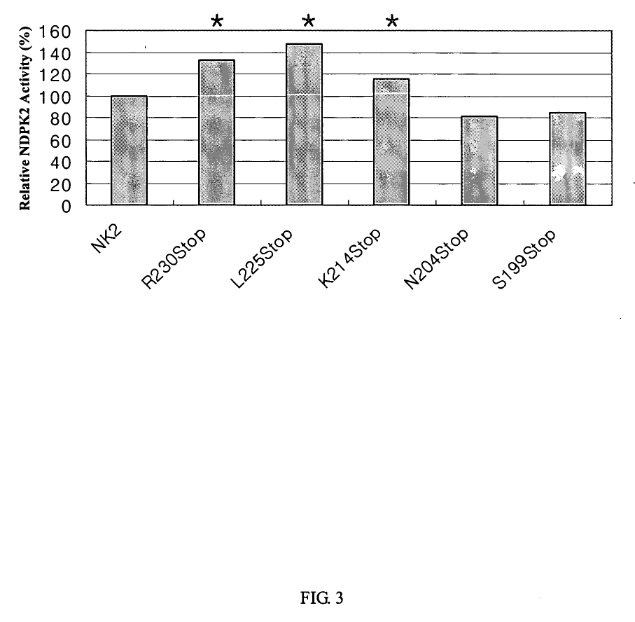

Therefore, from the study of these C-terminal deletion mutants, R230Stop, L225Stop and K214Stop were confirmed as hyperactive NDPK2 mutants. When their relative enzymatic activities were compared with wild-type NDPK2, the relative activities of R230Stop, L225Stop and K214Stop were 133%, 147% and 116%, respectively (FIG. 3).

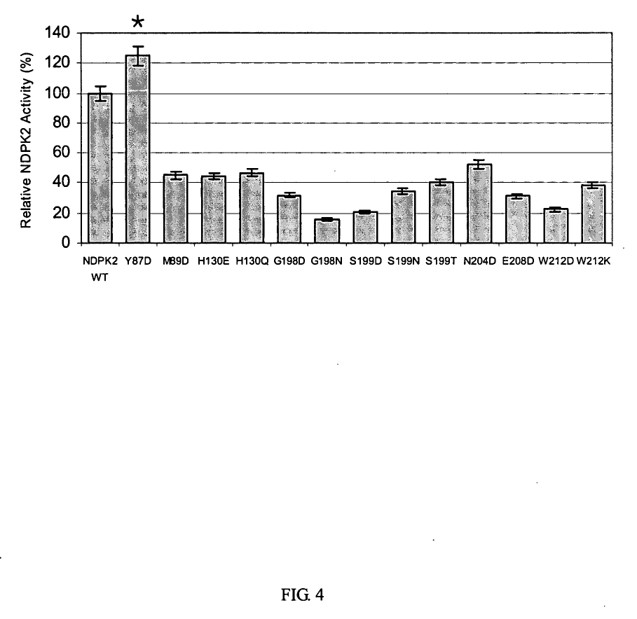

To get more hyperactive NDPK2, we tested the enzymatic activities of site-specific mutants of the active site H197-surrounding residues. H197-surrounding residue mutants were designed according to the known NDPK crystal structures, in which the residues near H197 were mutated to the charged residues. These selected residues include Y87 (SEQ ID NO: 5), M89, H130, G198, S199, N204, E208, and W212 that are very close to residue H197 in terms of the three dimensional structures. Results of the γ-phosphate exchange activity assay indicated that all mutations of selected residues affected NDPK2 activity, confirming that all the residues selected in the mutagenesis are structurally close to residue H197. Among these mutants, only mutant Y87D possessed a higher activity (125%) than the wild type (FIG. 4), whereas other mutants showed a significant decrease in their activities.

To further understand why these mutants are hyperactive, the pH-dependence of NDPK2 wild type and mutants was studied (Table 2). NDPK2's pKa₁ of 6.35 is believed to be mainly due to the active histidine residue H197. The pH range of 8.00-8.95 is the optimal pH condition for NDPK2-catalyzed γ-phosphate exchange reaction. NDPK2's pKa₂ of 8.95 is likely due to the other charged resides in the nucleotide-binding pocket, such as residues K91 and Y131. The pKa₁ values of the hypoactive mutants S199T (SEQ ID NO: 6) and E208D (SEQ ID NO: 7) are 6.50 and 7.00, respectively, which are higher than that of the wild type. In contrast, the hyperactive mutant Y87D has a lower pKa₁ value of 6.20. The pKa values of C-terminal deleted NDPK2 mutants were also determined. Results revealed that lower pKa values, especially lower pKa₁ values, correspond to higher NDPK activities (Table 2).

TABLE 2
The pKa Values Observed in the pH-dependence Profiles of NDPK2,
Pfr Phytochrome-Stimulated NDPK2, and NDPK2 Mutants.

| NDPKs | pKa1 | Optimal pH | pKa2 |
|---|---|---|---|
| NK2 WT (SEQ ID NO: 1) | 6.35 | 8.00-8.95 | 8.95 |
| L225Stop (SEQ ID NO: 3) | 6.10 | 7.85-8.90 | 8.90 |
| R230Stop (SEQ ID NO: 2) | 6.10 | 7.95-8.90 | 8.90 |
| Y87D (SEQ ID NO: 5) | 6.20 | 7.95-8.60 | 8.60 |
| S199T (SEQ ID NO: 6) | 6.50 | 8.20-8.80 | 8.80 |
| E208D (SEQ ID NO: 7) | 7.00 | 8.00-9.10 | 9.10 |

Therefore, we obtained three hyperactive NDPK2 (R230Stop, L225Stop, K214Stop) by C-terminal deletion and one hyperactive NDPK2 (Y87D) by site-directed mutagenesis. Since NDPK2 involves positively in the plant light and stress signal transduction, this invention enables us to develop transgenic plants with higher efficiency of light utilization and multiple stress tolerance, which can result in the increased yields of plants.

### EXAMPLES

All chemical reagents used were purchased from Sigma (St. Louis, Mo.) unless specified otherwise. Restriction and modifying enzymes were obtained from New England Biolabs, Inc. (Beverly, Mass.) and Roche Molecular Biochemicals (Indianapolis, Ind.). All polymerase chain reactions (PCR) were performed using high fidelity DNA polymerase, Turbo® Pfu polymerase which was purchased from Stratagene (La Jolla, Calif.).

Preparations of C-Terminal Deletion NDPK2 Mutants

Full-length Arabidopsis NDPK2 was prepared as previously described (Im et al., 2004). The C-terminal deletion mutants of NDPK2 were prepared by polymerase chain reactions using following primers: R230Stop (80-229aa) with 3′ primer: 5′-GAGACCCGGGC-TATAGCCATGTAGCTAGAGCCG-3′ (SmaI) (SEQ ID NO: 8); L225Stop (80-224aa) with 3′ primer: 5′-GAGACCCGGGCTA-AGCCGAATCCCACTTGC ATAGC-3′ (SmaI) (SEQ ID NO: 9); K214Stop (80-213aa) with 3′ primer: 5′-GAGACCC GGGCTAGA-ACCACAGACCAATCTCACG-3′ (SmaI) (SEQ ID NO: 10); N204Stop (80-203aa) with 3′ primer: 5′-GAGACCCGGGCTATTCA-GGGCTGTCACTACCATG-3′ (SmaI) (SEQ ID NO: 11); and S199Stop (80-198aa) with 3′ primer: 5′-GAGACCCGGGCTAACCA-TGCACAATGTTCCTTCC-3′ (SmaI) (SEQ ID NO: 12).

Preparations of Site-Specific NDPK2 Mutants by Site-Directed Mutagenesis

The in vitro mutagenesis of NDPK2 was performed using QuickChange™ site-directed mutagenesis protocol (Stratagene). The synthetic primers (sense) designed to produce the desired point mutations are listed as follows:

(SEQ ID NO: 13)

Y87D, 5′-GTTGAGGAGACTGACATTATGGTGAAACC-3′;

(SEQ ID NO: 14)

M89D, 5′-GGAGACTTACATTGACGT-GAAACCTGATGG-3′;

(SEQ ID NO: 15)

H130E, 5′-GAATTGGCTGAGGAGGAATATAAGGAGCTTAG-3′;

(SEQ ID NO: 16)

H130Q, 5′-GAATTGGCTGAGGAGCAATATAAGGAGCTTAG-3′;

(SEQ ID NO: 17)

P175S, 5′-TAGGGAAA-ACAGATTCG-CTTCAAGCTGAACC-3′;

(SEQ ID NO: 18)

H197C, 5′-GAAGGAACATGTGTGTGGTAGTG-ACAGC-3′;

(SEQ ID NO: 19)

G198D, 5′-GAACAT-TGTGCATGATAGTGACAGCCCTG-3′;

(SEQ ID NO: 20)

G198N, 5′-GAA-CATTGTGCATAATAGTGACAGCCCTG-3′;

(SEQ ID NO: 21)

S199D, 5′-CATTGTGCATGGTGATGACAG-CCCTGAAAAC-3′;

(SEQ ID NO: 22)

S199N, 5′-CATTGTGCATGGTAATGACAGCCCT-GAAAAC-3′;

(SEQ ID NO: 23)

S199T, 5′-CATTGTGCATGGTACTGACAGCCCTGAAAAC-3′;

(SEQ ID NO: 24)

N204D, 5′-GACAGCCCTGAA-GACGGCAAGCGTGAG-3′;

(SEQ ID NO: 25)

E208D, 5′-GAAAACGGCAAGCGTGACATTGGTCTGTGG-3′;

(SEQ ID NO: 26)

W212D, 5′-GTG-AGATTGGTCTGGACTTCAAAGAGGGC-3′;
and

(SEQ ID NO: 27)

W212K, 5′-GTGAGATTGGTCTGAAGTTCAAAGAGGGC-3′.

All primers were PAGE purified. The mutations were verified by DNA sequencing.
Purification of NDPK Proteins

Wild-type and mutant NDPK2s were subcloned into pGEX 4T vector (Pharmacia) using primers 5′-CTCGGATCCATGGAGGACGTTGAGGAGACTTAC-3′ (BamH1, forward) (SEQ ID NO: 28) and 5′-CGGAATTCTCACTCCCTTAGCCATGTAGC-3′ (EcoR1, backward) (SEQ ID NO: 29). NDPK proteins with a cleavable GST tag were expressed in Escherichia coli strain BL21 (DE3). The bacterial cells were induced for 4 h with 1 mM isopropyl β-D-thiogalactopyranoside (IPTG) at 310 K and then harvested by centrifugation at 4,500×g for 20 min. The cells were then resuspended in lysis buffer (1× PBS, phosphate-buffered saline) and lysed by sonication, after which the lysate was centrifuged at 16,000×g for 30 min. The resultant supernatant was applied to a glutathione-sepharose 4B affinity column pre-equilibrated with lysis buffer, after which the column was washed with ten bed volumes of lysis buffer. The GST fusion protein bound to the column was eluted with a buffer of 10 mM glutathione and 50 mM Tris-HCl (pH 8.0). GST Tags were cleaved from NDPKs by treatment with thrombin for 2 days at room temperature. The samples were then purified by size exclusion chromatography using a Superdex 200 column (Pharmacia Biotech) pre-equilibrated with a buffer of 50 mM NaCl and 10 mM Tris-HCl (pH 8.0), after which the fractions containing NDPKs were collected. GST protein remaining after the size exclusion chromatography was removed by glutathione-sepharose 4B affinity chromatography. Fractions containing NDPKs were then collected and used for assays.

In vitro Binding Assay

For the in vitro binding assay with ¹⁴C-labelled NDPK2, NDPK2 proteins were prepared by using the same method as that of unlabeled NDPK2 protein purification, with addition of 100 μCi L-methyl-¹⁴C methionine (Amersham) into 250 ml E. coli culture. The specific radioactivity of final ¹⁴C-labeled NDPK2 was around 600 cpm/μM. The radioactivity of the bound NDPK2 after immunoprecipitation with phytochromes was measured using a liquid scintillation counter (Beckman).

The in vitro binding assays of phytochrome and NDPK2 were performed by incubating 10 μg of phytochrome and 20 μg of NDPK2 in TBS buffer (50 mM Tris-HCl (pH 7.5), 150 mM NaCl), containing 5 mM MgCl₂, 2 mM dCDP, 0.1% NP40 and protease inhibitors, at 4° C. for 30 min. Either antibody Oat-22 against oat phyA or the specific antibody against NDPK2 was added to the reaction mixture and incubated for 40 min. The antibody-protein complexes were recovered by incubation with 0.1 volumes of Protein A/G beads (Oncogene) for an additional 30 min, and then collected by centrifugation. Beads were washed five times in TBS buffer. The attached proteins were solubilized in 1× SDS sample buffer at 100° C. for 5 min and then resolved on 12% (w/v) SDS-polyacrylamide gels, followed by transferring to polyvinylidene difluoride (PVDF) membranes for western blotting analysis.

NDPK2 γ-Phosphate Exchange Activity Assay

NDPK2 γ-phosphate exchange activity was measured as previously described (20) with minor modifications. The assay buffer contained 50 mM Tris-HCl (pH 7.5), 5 mM MgCl₂, 3 mM phosphoenolpyruvate, 2 mM ATP, 0.3 mM NADH, 5 units pyruvate kinase (PK), 5 units lactate dehydrogenase (LDH), and 1 mM dCDP. The reaction was initiated by adding 3 nM NDPK2. NDPK2 activity was measured by monitoring the LDH-PK-coupled NADH decrease at 340 nm. The phytochrome effect was examined by incubating a mixture of native oat phyA and NDPK2 under illumination of red light (660 nm, Pfr form) or far-red light (730 nm, Pr form) for 8 min and measuring NDPK2 activity. The Kₘ values of NDPK2 with different nucleotides were measured in a similar manner with a fixed concentration of 60 nM of Pfr phytochrome. For pH-dependence NDPK2 activity measurements, AMT isoionic buffer (50 mM acetic acid, 50 mM Mes and 100 mM Tris-HCl) was used in a pH range of 5.0-9.5.

### REFERENCES

Choi, G Yi, H., Lee, J., Kwon, Y-K., Soh, M.-S., Shin, B., Luka, Z., Hahn, T.-R., and Song, P-S. (1999) Phytochrome signaling is mediated through nucleoside diphosphate kinase 2. Nature 401, 610-613.
Im, Y-J., Kim, J-I., Shen, Y, Na, Y., Han, Y-J., Kim, S-H., Song, P-S., and Eom, S. H. (2004) Structural analysis of Arabidopsis thaliana nucleoside diphosphate kinase-2 for plant phytochrome signaling. J. Mol. Biol. 343, 659-670.
Moon, H., Lee, B., Choi, G., Shin, D., Prasad, D. T., Lee, O., Kwak, S.-S., Kim, D. H., Nam, J., Bahk, J., Hong, J. C., Lee, S. Y., Cho, M. J., Lim, C. O., & Yun, D. J. (2003). NDP kinase 2 interacts with two oxidative stress-activated MAPKs to regulate cellular redox state and enhances multiple stress tolerance in transgenic plants. Proc. Natl Acad. Sci. USA, 100, 358-363.
Shen, Y., Kim, J-I., and Song, P-S. (2005) NDPK2 as a signal transducer in the phytochrome-mediated light signaling. J. Biol. Chem. 280, 5740-5749.

What is claimed is:

1. A nucleic acid molecule encoding modified Nucleoside Di-Phosphate Kinase 2 (NDPK2) protein comprising:
i) R230Stop modified protein of SEQ ID NO: 2;
ii) L225Stop modified protein of SEQ ID NO: 3;
iii) K214Stop modified protein of SEQ ID NO: 4; and
iv) Y87D modified protein of SEQ ID NO: 5,
wherein its enzymatic activity has been increased to confer higher efficiency of light utilization and stress tolerance in vivo.

2. An expression vector for transformation of plant cells comprising:
i) a polynucleotide of claim 1 encoding a modified NDPK2; and
ii) regulatory sequences operatively linked to the polynucleotide such that the polynucleotide is expressed in the plant cell,
wherein said expression results in higher efficiency of light utilization and stress tolerance.

3. A transgenic plant cell transformed with the expression vector of claim 2.

4. A transgenic plant in higher efficiency of light utilization and stress tolerance grown from the transgenic plant cell of claim 3.


SEQUENCE LISTING (29 sequences)

SEQ ID NO: 1 - protein, 231 aa, Arabidopsis thaliana
MVGATVVSKWTPLCVASPPERNSASLNPHCSPARVNFRTALAAFRPQFRLFSRNSASRRRLRASSSAESGIFLPHLVASMEDVEETYIMVKPDGIQRGLVGEIISRFEKKGFKLIGLKMFQCPKELAEEHYKELSAKSFFLTLIEYITSGPVVCMAWEGVGVVASARKLIGKTDPLQAEPGTIRGDLAVQTGRNIVHGSDSPENGKREIGLWFKEGELCKWDSALATWLRE

SEQ ID NO: 2 - protein, 229 aa, Arabidopsis thaliana
MVGATVVSKWTPLCVASPPERNSASLNPHCSPARVNFRTALAAFRPQFRLFSRNSASRRRLRASSSAESGIFLPHLVASMEDVEETYIMVKPDGIQRGLVGEIISRFEKKGFKLIGLKMFQCPKELAEEHYKELSAKSFFLTLIEYITSGPVVCMAWEGVGVVASARKLIGKTDPLQAEPGTIRGDLAVQTGRNIVHGSDSPENGKREIGLWFKEGELCKWDSALATWL

SEQ ID NO: 3 - protein, 224 aa, Arabidopsis thaliana
MVGATVVSKWTPLCVASPPERNSASLNPHCSPARVNFRTALAAFRPQFRLFSRNSASRRRLRASSSAESGIFLPHLVASMEDVEETYIMVKPDGIQRGLVGEIISRFEKKGFKLIGLKMFQCPKELAEEHYKELSAKSFFLTLIEYITSGPVVCMAWEGVGVVASARKLIGKTDPLQAEPGTIRGDLAVQTGRNIVHGSDSPENGKREIGLWFKEGELCKWDSA

SEQ ID NO: 4 - protein, 213 aa, Arabidopsis thaliana
MVGATVVSKWTPLCVASPPERNSASLNPHCSPARVNFRTALAAFRPQFRLFSRNSASRRRLRASSSAESGIFLPHLVASMEDVEETYIMVKPDGIQRGLVGEIISRFEKKGFKLIGLKMFQCPKELAEEHYKELSAKSFFLTLIEYITSGPVVCMAWEGVGVVASARKLIGKTDPLQAEPGTIRGDLAVQTGRNIVHGSDSPENGKREIGLWF

SEQ ID NO: 5 - protein, 231 aa, Arabidopsis thaliana
MVGATVVSKWTPLCVASPPERNSASLNPHCSPARVNFRTALAAFRPQFRLFSRNSASRRRLRASSSAESGIFLPHLVASMEDVEETDIMVKPDGIQRGLVGEIISRFEKKGFKLIGLKMFQCPKELAEEHYKELSAKSFFLTLIEYITSGPVVCMAWEGVGVVASARKLIGKTDPLQAEPGTIRGDLAVQTGRNIVHGSDSPENGKREIGLWFKEGELCKWDSALATWLRE

SEQ ID NO: 6 - protein, 231 aa, Arabidopsis thaliana
MVGATVVSKWTPLCVASPPERNSASLNPHCSPARVNFRTALAAFRPQFRLFSRNSASRRRLRASSSAESGIFLPHLVASMEDVEETYIMVKPDGIQRGLVGEIISRFEKKGFKLIGLKMFQCPKELAEEHYKELSAKSFFLTLIEYITSGPVVCMAWEGVGVVASARKLIGKTDPLQAEPGTIRGDLAVQTGRNIVHGTDSPENGKREIGLWFKEGELCKWDSALATWLRE

SEQ ID NO: 7 - protein, 231 aa, Arabidopsis thaliana
MVGATVVSKWTPLCVASPPERNSASLNPHCSPARVNFRTALAAFRPQFRLFSRNSASRRRLRASSSAESGIFLPHLVASMEDVEETYIMVKPDGIQRGLVGEIISRFEKKGFKLIGLKMFQCPKELAEEHYKELSAKSFFLTLIEYITSGPVVCMAWEGVGVVASARKLIGKTDPLQAEPGTIRGDLAVQTGRNIVHGSDSPENGKRDIGLWFKEGELCKWDSALATWLRE

SEQ ID NO: 8 - DNA, 33 bp, Artificial
  Primer
gagacccgggctatagccatgtagctagagccg

SEQ ID NO: 9 - DNA, 35 bp, Artificial
  Primer
gagacccgggctaagccgaatcccacttgcatagc

SEQ ID NO: 10 - DNA, 34 bp, Artificial
  Primer
gagacccgggctagaaccacagaccaatctcacg

SEQ ID NO: 11 - DNA, 34 bp, Artificial
  Primer
gagacccgggctattcagggctgtcactaccatg

SEQ ID NO: 12 - DNA, 34 bp, Artificial
  Primer
gagacccgggctaaccatgcacaatgttccttcc

SEQ ID NO: 13 - DNA, 29 bp, Artificial
  Primer
gttgaggagactgacattatggtgaaacc

SEQ ID NO: 14 - DNA, 30 bp, Artificial
  Primer
ggagacttacattgacgtgaaacctgatgg

SEQ ID NO: 15 - DNA, 32 bp, Artificial
  Primer
gaattggctgaggaggaatataaggagcttag

SEQ ID NO: 16 - DNA, 32 bp, Artificial
  Primer
gaattggctgaggagcaatataaggagcttag

SEQ ID NO: 17 - DNA, 31 bp, Artificial
  Primer
tagggaaaacagattcgcttcaagctgaacc

SEQ ID NO: 18 - DNA, 28 bp, Artificial
  Primer
gaaggaacatgtgtgtggtagtgacagc

SEQ ID NO: 19 - DNA, 29 bp, Artificial
  Primer
gaacattgtgcatgatagtgacagccctg

SEQ ID NO: 20 - DNA, 29 bp, Artificial
  Primer
gaacattgtgcataatagtgacagccctg

SEQ ID NO: 21 - DNA, 31 bp, Artificial
  Primer
cattgtgcatggtgatgacagccctgaaaac

SEQ ID NO: 22 - DNA, 31 bp, Artificial
  Primer
cattgtgcatggtaatgacagccctgaaaac

SEQ ID NO: 23 - DNA, 31 bp, Artificial
  Primer
cattgtgcatggtactgacagccctgaaaac

SEQ ID NO: 24 - DNA, 27 bp, Artificial
  Primer
gacagccctgaagacggcaagcgtgag

SEQ ID NO: 25 - DNA, 30 bp, Artificial
  Primer
gaaaacggcaagcgtgacattggtctgtgg

SEQ ID NO: 26 - DNA, 29 bp, Artificial
  Primer
gtgagattggtctggacttcaaagagggc

SEQ ID NO: 27 - DNA, 29 bp, Artificial
  Primer
gtgagattggtctgaagttcaaagagggc

SEQ ID NO: 28 - DNA, 33 bp, Artificial
  Primer
ctcggatccatggaggacgttgaggagacttac

SEQ ID NO: 29 - DNA, 29 bp, Artificial
  Primer
cggaattctcactcccttagccatgtagc